(19) United States
(12) Patent Application Publication
(10) Pub. No.: US 2023/0390539 A1
(43) Pub. Date: Dec. 7, 2023

(54) INTRODUCER AND DILATOR FOR SURGICAL INSTRUMENT

(71) Applicant: Armen Gregorian, Glendale, CA (US)

(72) Inventor: Armen Gregorian, Glendale, CA (US)

(21) Appl. No.: 18/325,761

(22) Filed: May 30, 2023

Related U.S. Application Data
(60) Provisional application No. 63/348,617, filed on Jun. 3, 2022.

(51) Int. Cl. A61M 29/00; A61B 17/068

(52) U.S. Cl. CPC A61M 29/00; A61B 17/068

(57) ABSTRACT

In some embodiments, a device includes a first portion and a second portion. The first portion includes a rounded shape for introduction into an opening of a structure. The second portion includes a recess to receive a needle from an instrument. A retrieval mechanism retrieves the device after removal of the device from the instrument. The device is collapsible in the structure when removed from the instrument after insertion of the instrument and the device into the structure.

### CROSS REFERENCE TO RELATED APPLICATIONS

Pursuant to 35 U.S.C. § 119(e), this application is entitled to and claims the benefit of the filing date of U.S. Provisional App. No. 63/348,617 filed Jun. 3, 2022, entitled “COMBINED SURGICAL STAPLER AND DILATOR DEVICE AND METHODS OF USE THEREOF”, the content of which is incorporated herein by reference in its entirety for all purposes.

### BACKGROUND

Surgical stapling instruments typically have a mechanism for firing a plurality of staples from a staple holding cartridge to an anvil positioned opposite the staple cartridge against which the staples are formed. In use, the surgeon will place tissue between the staple cartridge and anvil and, by firing the instrument, cause the staples to become clenched to the tissue.

Prior to inserting the surgical stapler for use, mobilization of a segment of the intestine must be accomplished. Mobilization is achieved by freeing the intestine from its cavity attachment and is then followed by separating its blood supply. The mobilization can be conducted laparoscopically or through opening the peritoneal cavity. Following mobilization, resection of the segment to be removed must follow. The resection of the segment is removed and the remaining ends of the bowel are anastomized to guarantee the continuity of the intestinal track. In an intestinal end-to-end anastomosis, a portion of the intestinal tract is removed and the remaining ends are rejoined by using a circular stapler.

In order to ease the procedure and create a larger cavity in the rectum for which to operate, in the United States and most places in the world, the rectum is dilated prior to introducing the blunt tip stapler for an End to End Anastomosis (EEA). Several apparatus and procedures have developed for the stapler which is a gun that is introduced into the anus and blindly maneuvered through the rectum prior to introducing the stapler, which has a blunt circular tip. A separate dilator/sizer is frequently used to dilate the rectum and then removed from the rectum prior to inserting and advancing the stapler through the rectum. The dilators are generally metal instruments that are reusable, and need to be sterilized by auto clave devices, and stored for a dilation procedure.

To join the tubular structures after dilation, one end of the intestine is secured about an anvil and the other end of the intestine is held in place adjacent a staple cartridge. Preferably, the anvil has a shaft that is removably connected to the instrument. Once the anvil shaft is secured to the instrument, the anvil is drawn into close approximation to the stapling cartridge. The instrument is then fired to cause the staples to pass through tissue of both organs and become formed against the anvil. During the firing step, a circular knife is advanced to cut tissue inside the staple line, thereby establishing a passage between the organs. After firing, the instrument is typically removed by withdrawing the anvil through the staple line, after which the surgeon will carefully inspect the surgical site to ensure a proper anastomosis (joining) has been achieved.

### BRIEF DESCRIPTION OF THE DRAWINGS

The included drawings are for illustrative purposes and serve only to provide examples of possible structures and operations for the disclosed inventive systems, apparatus, methods and computer program products. These drawings in no way limit any changes in form and detail that may be made by one skilled in the art without departing from the spirit and scope of the disclosed implementations.

FIGS. 1A to 1F depict an example of a device according to some embodiments.

FIG. 2A depicts another example of a device according to some embodiments.

FIG. 2B depicts a side perspective of the device according to some embodiments.

FIG. 3 depicts a cross-section of device according to some embodiments.

FIGS. 4A to 4D depict additional examples of device 100 according to some embodiments.

FIGS. 5A and 5B depict an example of a stapler according to some embodiments.

FIG. 6 depicts an example of a stapler with a needle extended according to some embodiments.

FIG. 7 depicts an example of the device being inserted into the needle according to some embodiments.

FIG. 8 depicts an example of the device in which it has been attached to a stapler according to some embodiments.

FIG. 9A depicts the stapler and the device that have been attached and can be introduced into a structure.

FIG. 9B shows the insertion of the device and the stapler into the structure according to some embodiments.

FIG. 9C depicts an example of the device being separated from the stapler according to some embodiments.

FIG. 9D shows the retrieval of the device according to some embodiments.

FIG. 9E depicts the stapling of the structures according to some embodiments.

FIG. 9F shows an example of the stapled structure according to some embodiments.

FIG. 9G depicts an example of the stapler and the device after removal according to some embodiments.

FIG. 10 depicts a simplified flow chart of a method for performing the stapling of structures according to some embodiments.

### DETAILED DESCRIPTION

Described herein are techniques for a dilator and introducer system. In the following description, for purposes of explanation, numerous examples and specific details are set forth to provide a thorough understanding of some embodiments. Some embodiments as defined by the claims may include some or all the features in these examples alone or in combination with other features described below, and may further include modifications and equivalents of the features and concepts described herein.

Overview

A device may be used as an introducer and a dilator for a procedure on a subject, such as a procedure that may staple the tissue of two structures together during a surgery. As discussed above, the device may be used during an end-to-end anastomosis, but can be used in other procedures, such as a laparoscopic gastric bypass surgery, or other procedures that require introduction of another device and dilation, and not necessarily stapling two structures together. Various examples of the instruments used in the procedure include a stapler, such as a linear stapler, which typically applies parallel rows of staples, and a circular stapler, which forms a concentric or circular rows of staples. Other types of staplers may also be appreciated. The device may attach to the tip of the stapler. In some embodiments, the device may attach onto a retractable needle of the stapler. The device may then provide the combination of the introduction into and the advancement of the stapler through a structure in addition to dilation. The dilation may slightly expand the structure and allow for the advancement of the stapler into the structure. Once reaching the desired position inside of the structure, the device may be removed from the stapler. For example, the needle may be retracted into the stapler, which releases the device from the needle of the stapler, but other methods of releasing the device may also be appreciated.

Once the device is released from the stapler, a mechanism to retrieve the device may be used, such as a string may be used to retrieve the device. The device may be collapsible to allow for the device to be retrieved while the stapler is still inserted in the structure. As the structure is retrieved and pulled to the side of the stapler, the structure may collapse, which allows the device to slide past the end of the stapler to the side of the stapler. The device may be left in between the stapler and the side of the structure. Then, the stapler may be used to staple the two structures together. For example, as discussed above, to join the tubular structures after dilation, one end of the intestine is secured about an anvil and the other end of the intestine is held in place adjacent a staple cartridge. Once the anvil shaft is secured to the stapler, the anvil is drawn into close approximation to the stapling cartridge. The stapler is then fired to cause the staples to pass through tissue of both intestines and become formed against the anvil. During the firing step, a circular knife is advanced to cut tissue inside the staple line, thereby establishing a passage between the organs. After firing, the stapler is removed by withdrawing the anvil through the staple line. Also, the device may be removed using the string. For example, the stapler may be removed first, and then a string may be used to remove the device, or vice versa.

Device

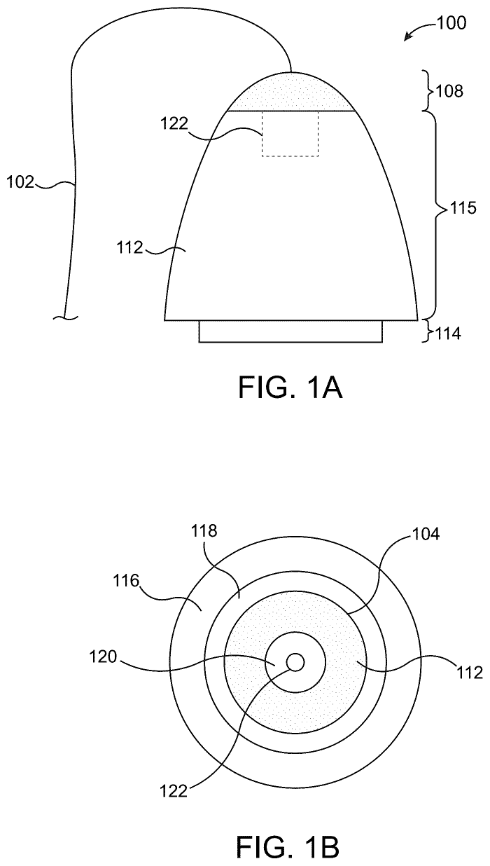

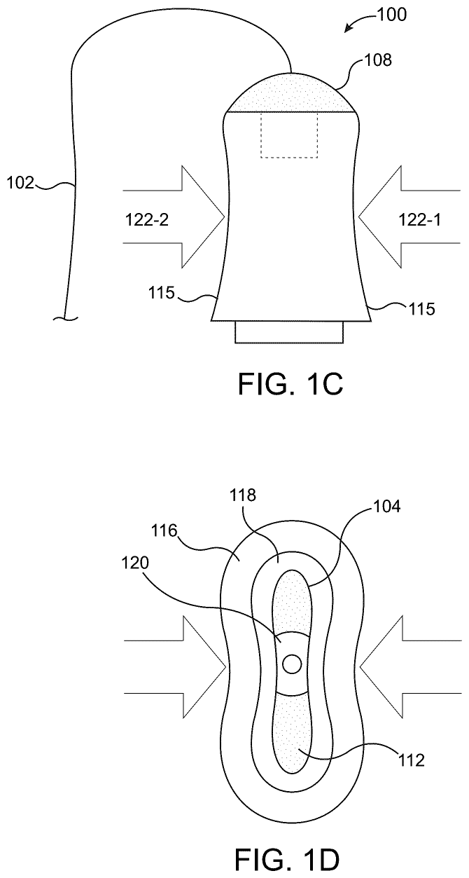

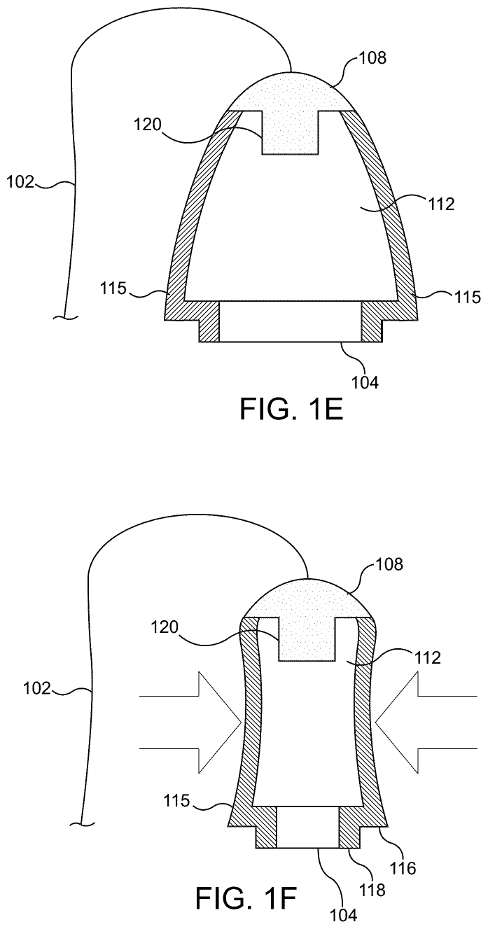

FIGS. 1A to 1F depict an example of a device 100 according to some embodiments. In FIGS. 1A to 1F, device 100 is detached from an instrument (e.g., a stapler). Although device 100 is described as being attachable to a stapler, device 100 may be attached to other instruments that require introduction and dilation. The device includes a retrieval mechanism, such as a string 102, but other retrieval devices may be used. In FIG. 1A, in some embodiments, string 102 is inserted in a distal end 108 of device 100. For example, string 102 may be used to retrieve device 100 once it has been detached from the stapler. String 102 may be attached through an inner structure 122 through a channel (not shown). Other methods of attaching string 102 to device may be appreciated, such as adhering string 102 to distal end 108 of device 100.

Distal end 108 of device 100 may be a first portion that may have a wall with a rounded edge. The rounded edge may facilitate introduction of device 100 into a structure, such as the rectum and an intestine. The first portion may also be other shapes, such as having two rounded edges that meet at a tip. Device 100 may perform dilation of a structure when inserted into the structure. The dilation may increase the size of the structure as device 100 moves through the structure because the diameter of device 100 may be larger than the diameter of the structure. The distal end 108 of device 100 may be made of a first type of material to allow for the introduction of the stapler into a structure. In some embodiments, the first type of material may be more dense than a second type of material that is located in the side area 115. For example, if silicon is being used, the material of distal end 118 may be a more dense version of silicon than the silicon on proximal end 114. The less dense material of device 100 at side area 115 may be made of a softer material to allow device 100 to collapse. The material of device 100 may also not bruise or damage the side of the structure. Device 100 includes a cavity 112 inside of device 100. Cavity may be an opening inside of the walls of device 100. Cavity 112 and the softer material allow device 100 to collapse when force is applied to the side of device, such as via a side wall of the intestine and the stapler. The collapsing of device 100 may make the diameter of device 100 smaller to allow it to be moved to a side of the stapler (or fully retrieved) while the stapler is still inserted into the structure.

Proximal end 114 may be a second portion that may include a recess 104 that allows an attachment to the stapler. Recess 104 may be an opening. Also, proximal end may include a ledge 114 and ridge 118 that form the outside of recess 104. Cavity 112 is located through recess 104 in device 100. An inner structure 120 may support distal end 108 and be made of the same material of distal end 118. Inner structure 120 may include a channel 122 through which, string 104 may be inserted, such as through a proximal end 114 of device 100 to the distal end 108. A stopper may attach string 102 to device 100 to allow device 100 to be pulled and retrieved using string 102. For example, the attachment to the stapler may be made by inserting a needle of the stapler into recess 104. However, other methods of attaching device 100 to the stapler may be used, such as a tongue and groove attachment, suction, magnetic coupling, or other fastening methods. Proximal end 114 may also have a portion that is insertable into the stapler, which will be discussed in more detail below. The removal of device 100 from the stapler may also be performed by retracting the needle inside of the stapler, which causes device 100 to detach from the needle and the stapler.

FIG. 1C depicts the collapsibility of device 100 according to some embodiments. At 122-1 and 122-2, force may be applied to the side areas 115 of device 100. The less dense material of side area 115 allows it to collapse in the direction of the force. The more dense material of distal end 108 may let it keep its rounded shape.

FIG. 1D depicts a bottom view of device 100 when collapsed according to some embodiments. Ledge 116 and ridge 118 may be collapsed and form an oval shape. Inner structure 120 is not collapsed and maintains its shape. Recess 104 and cavity 112 are also collapsed.

FIG. 1E depicts a cross sectional view of device 100 according to some embodiments. Cavity 112 is shown inside of device 100 and recess 104 shows an opening at proximal end 114. Walls of side area 115 are shown in addition to inner structure 120 at distal end 108.

FIG. 1F depicts a cross sectional view of device 100 when collapsed according to some embodiments. Ledge 116 and ridge 118 may be collapsed. Inner structure 120 is not collapsed and maintains its shape. Recess 104 and cavity 112 are also collapsed.

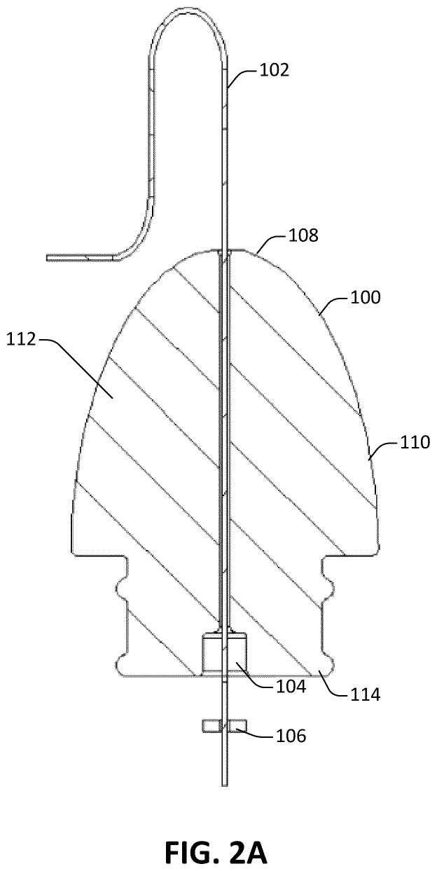

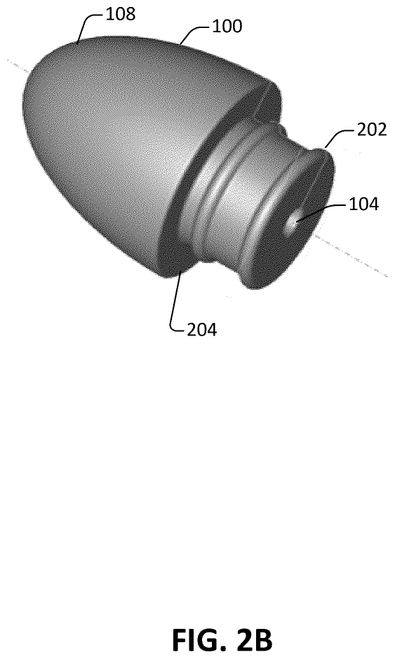

FIG. 2A depicts another example of a device 100 according to some embodiments. Device 100 is detached from an instrument (e.g., a stapler) in this figure. Although device 100 is described as being attachable to a stapler, device 100 may be attached to other instruments that require introduction and dilation. The device includes a retrieval mechanism, such as a string 102, but other retrieval devices may be used. In some embodiments, string 102 is inserted through a recess 104 in device 100 and through a channel, such as through a proximal end 114 of device 100 to the distal end 108. Recess 104 may be an opening. The channel may be part of a cavity inside of device 100. A stopper 106 may attach string 102 to device 100 to allow device 100 to be pulled and retrieved using string 102. For example, string 102 may be used to retrieve device 100 once it has been detached from the stapler. Other methods of attaching string 102 to device may be appreciated, such as adhering string 102 to distal end 108 of device 100.

A distal end 108 of device 100 may have a wall with a rounded edge. The rounded edge may facilitate introduction of device 100 into a structure, such as the rectum and an intestine. Device 100 may perform dilation of a structure when inserted into the structure. The dilation may increase the size of the structure as device 100 moves through the structure because the diameter of device 100 may be larger than the diameter of the structure. The material of device 100 may also not bruise or damage the side of the structure.

Proximal end 114 may include a recess 104 that allows an attachment to the stapler. For example, the attachment to the stapler may be made by inserting a needle of the stapler into recess 104. However, other methods of attaching device 100 to the stapler may be used, such as a tongue and groove attachment, suction, magnetic coupling, or other fastening methods. Proximal end 114 may also have a portion that is insertable into the stapler, which will be discussed in more detail below. The removal of device 100 from the stapler may also be performed by retracting the needle inside of the stapler, which causes device 100 to detach from the needle and the stapler.

FIG. 2B depicts a side perspective of device 100 according to some embodiments. Recess 104 is shown as a circular structure that a needle of the stapler can be inserted. Also, the proximal end 114 may include a rounded structure 202. The rounded structure may fit into a circular stapler blunt end. The diameter of the rounded structure 202 may be less than the diameter of a ledge 204 to allow rounded structure 202 to be inserted into the end of the stapler. In some embodiments, device 100 in FIGS. 2A and 2B may not include a cavity other than the channel.

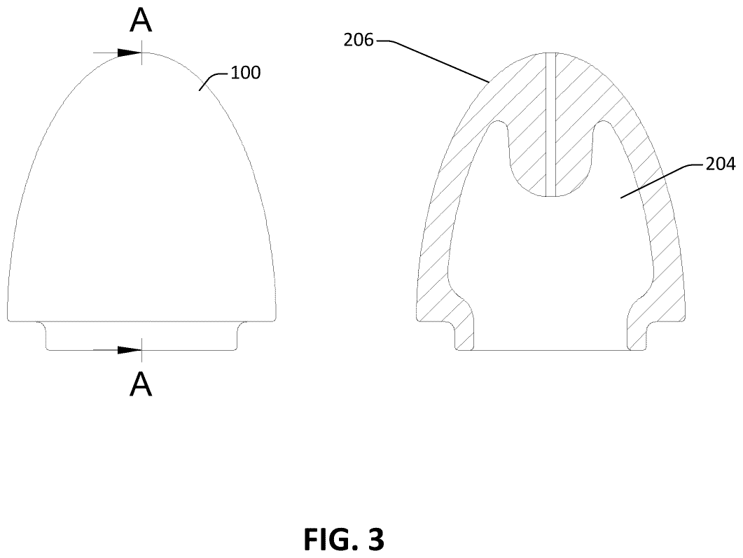

FIG. 3 depicts a cross-section of device 100 according to some embodiments. Device 100 includes a more dense material inside of it at 204. The outside walls 206 may be made of a less dense material to allow for device 100 to collapse.

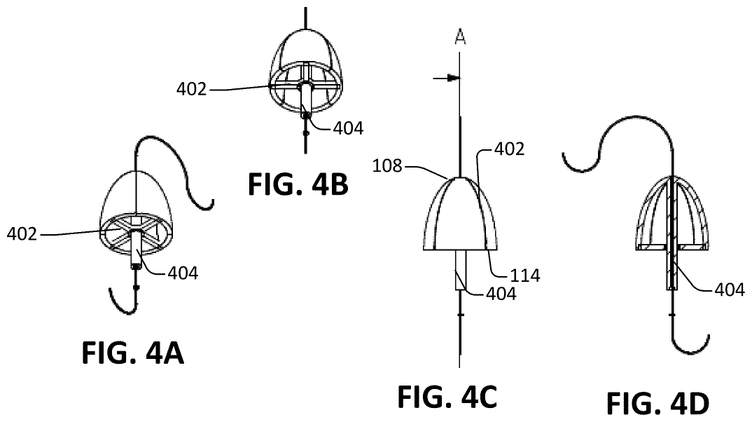

FIGS. 4A to 4D depict additional examples of device 100 according to some embodiments. As discussed above, device 100 may be collapsible to allow for easier removal during procedures. In some embodiments, leaflets 402 may be used to facilitate the collapsibility of device 100.

Leaflets 402 may be attached to the outside of device 100 and to a center stud 404. Leaflets may be a rectangular shape that attach to the edge of device 100 and center stud 404. In some embodiments, four leaflets are included, but other numbers of leaflets may be included, such as 2, 3, 4, 5, etc. Leaflets 402 may be made of a flexible material that may support the outside of device 100 as it is being introduced into the structure. Also, leaflets 402 may be made of a first type of material that is flexible to allow for the collapsing of device 100 when force is applied to the outside of device 100. For example, leaflets may be a less dense silicon compared to the silicon used in other areas of device 100.

Leaflets 402 may start in an extended position, which is used for the introduction of the stapler into the structure and also a dilation of the structure. Then, in a collapsed position, leaflets 402 collapse device 100 to allow for easier retrieval of device 100 from the structure. Leaflets may be configured in different ways. For example, leaflets 402 may be attached to center stud 404 and walls of device 100 as shown in FIG. 4A and FIG. 4B. Also, leaflets 402 may be attached to the distal end 108 to the proximal end 114 in FIG. 4C. Or, both configurations of leaflets 402 may be used in combination as shown in FIG. 4D which includes the leaflets of FIGS. 4A/4B, and 4C. The leaflets may collapse when force is applied inward. This will collapse the sections of structure of device 100 that are made of the more dense material (e.g., silicon). Although a mechanical enactment device is described, other methods may be used to collapse device 100 including an inflatable balloon, torsional coil springs, mnemonic enactment, and other methods. Is noted that the material of device 100 also enables the collapsible feature of device 100 in addition to or in lieu of the use of leaflets 402.

The following will now describe the use of device 100 with an instrument, such as a stapler.

Stapler

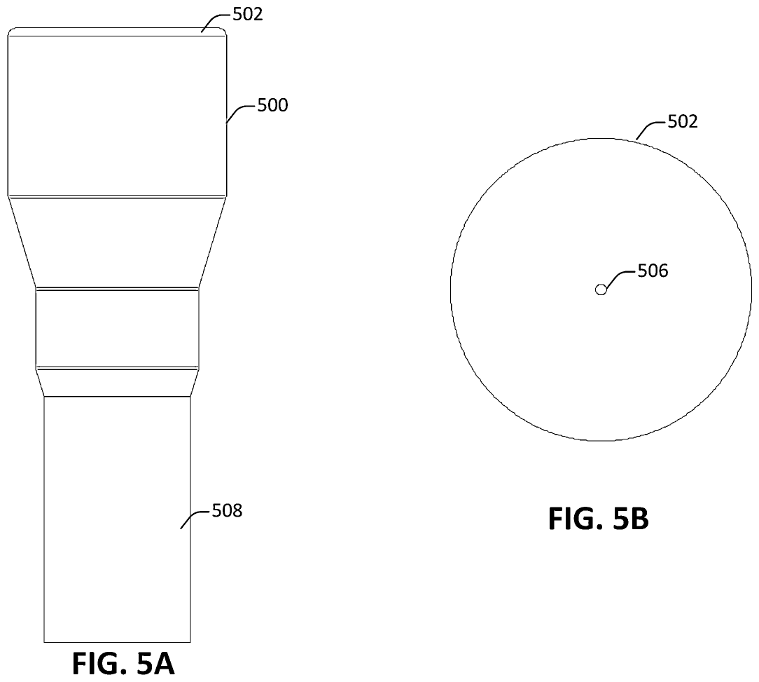

FIGS. 5A and 5B depict an example of a stapler 500 according to some embodiments. Stapler 500 may be an instrument that performs stapling, but devices with other functions may be used. Stapler 500 may also include a knife to cut tissue. Stapler 500 includes a handle 508 at a proximal end and an opening 502 at the distal end. Handle 408 may be used to hold stapler 500 and include controls to perform stapling, and also extend or retract a needle, which will be described below.

Device 100 may be inserted into opening 502. Opening 502 may be circular and also include a stapling cartridge that can perform stapling in a circular structure, such as staple two structures together. Opening 502 is shown in FIG. 5B in more detail. The opening may be circular to perform stapling in a circular structure, but other shapes may be appreciated, such as a linear shape may be used to staple in a line instead of a circular structure. A needle 506 is shown that can be extended or retracted.

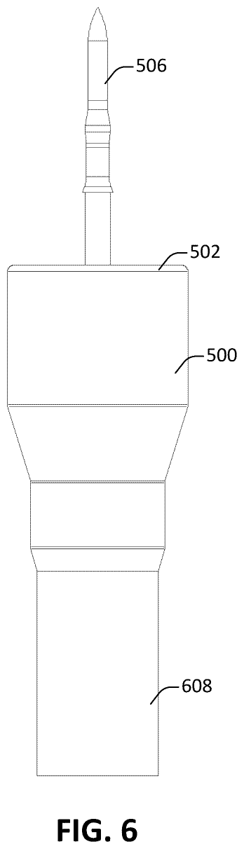

FIG. 6 depicts an example of stapler 500 with needle 506 extended according to some embodiments. Needle 506 has been extended from opening 502, such as using a mechanism on handle 508. Needle 506 may be made from a hard material, such as a metal. As will be discussed in more detail below, needle 506 may be coupled to (e.g., inserted into) an anvil to perform the stapling of two structures.

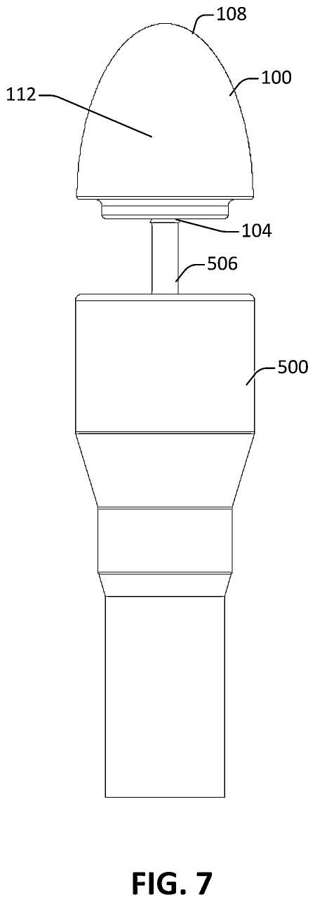

Device 100 may be configured to attach to needle 506 through recess 104. FIG. 7 depicts an example of device 100 being inserted into needle 506 according to some embodiments. As shown, the proximal end of device 100 has been inserted into needle 506 through recess 104. Device 100 becomes attached to needle 506. During attachment, needle 506 may proceed through cavity 112 towards the distal end 108 of device 100. Device 100 is then attached to stapler 500 via needle 506.

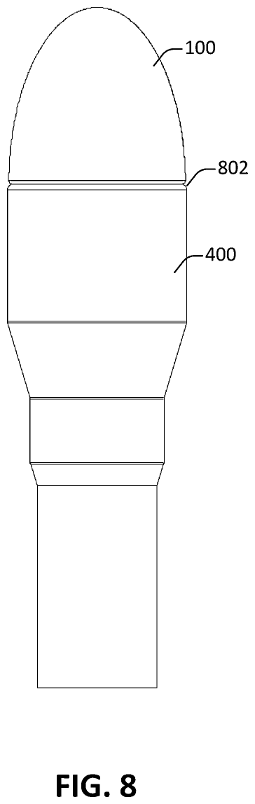

FIG. 8 depicts an example of device 100 in which it has been attached to a stapler 500 according to some embodiments. At 802, the distal end of stapler device 100 is coupled to the proximal end of stapler 500. In some embodiments, device 100 may be inserted onto needle 506 and couples to the distal end of stapler 500. In other embodiments, needle 506 may be slightly retracted to move the proximal end of device 100 to be coupled to stapler 500.

Device 100 may cover the opening or blunt end of stapler 500. The outside of device 100 may be a similar diameter to the outside of stapler 500. The diameter of device 100 may also be larger than the diameter of stapler 500. The shape of device 100 may allow for the introduction of stapler 500 without damaging the walls of the structure. Additionally, the distal tip of device 100 may dilate the structure without damaging the structure or getting caught on any folds of the structure. Device 100 is an introducer that introduces stapler up the rectal stump and it dilates while maneuvering through the rectum. This is very important as the rectum has valves of Houston, which the dilator is able to open as it is advancing.

Use of Stapler and Device During a Procedure

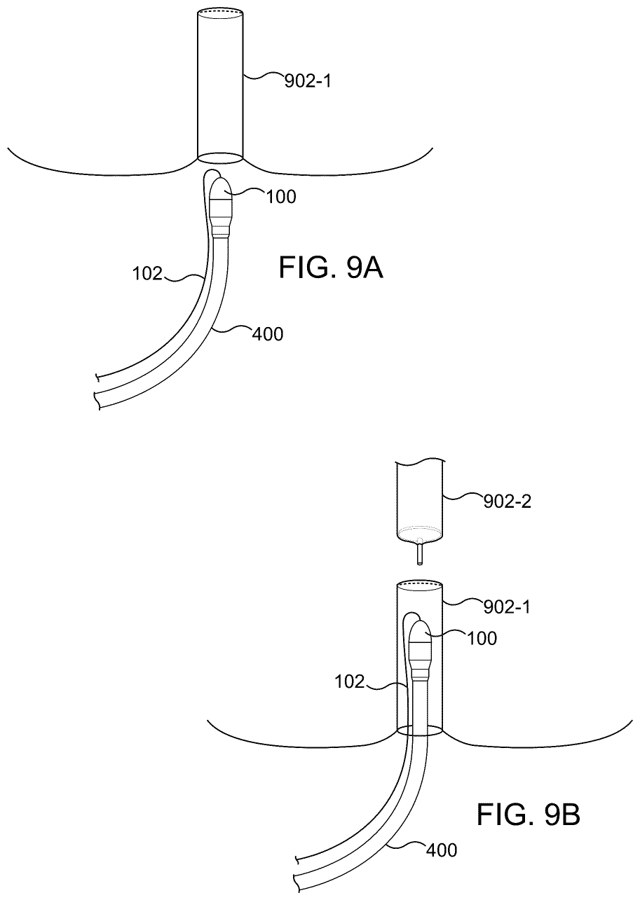

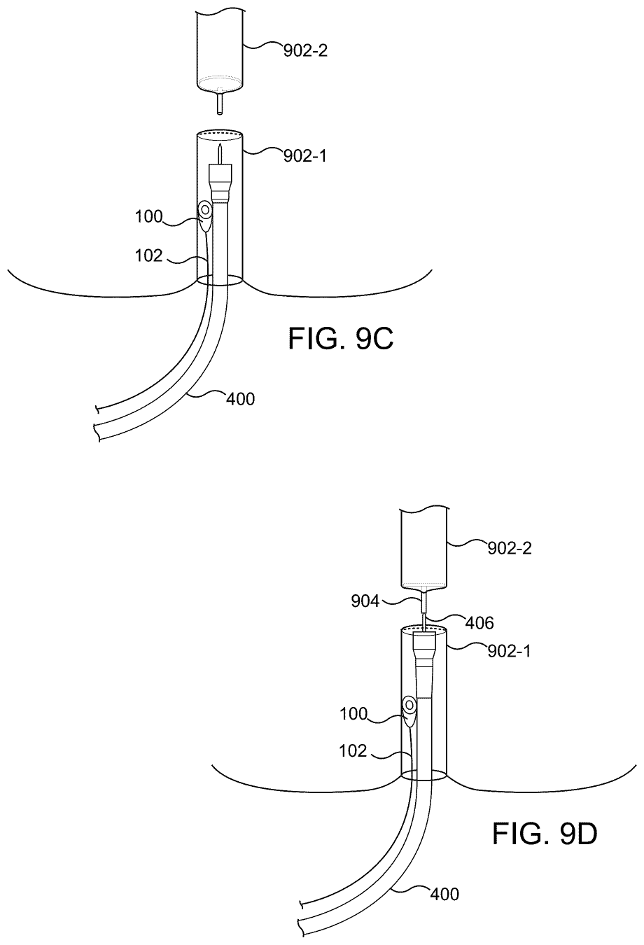

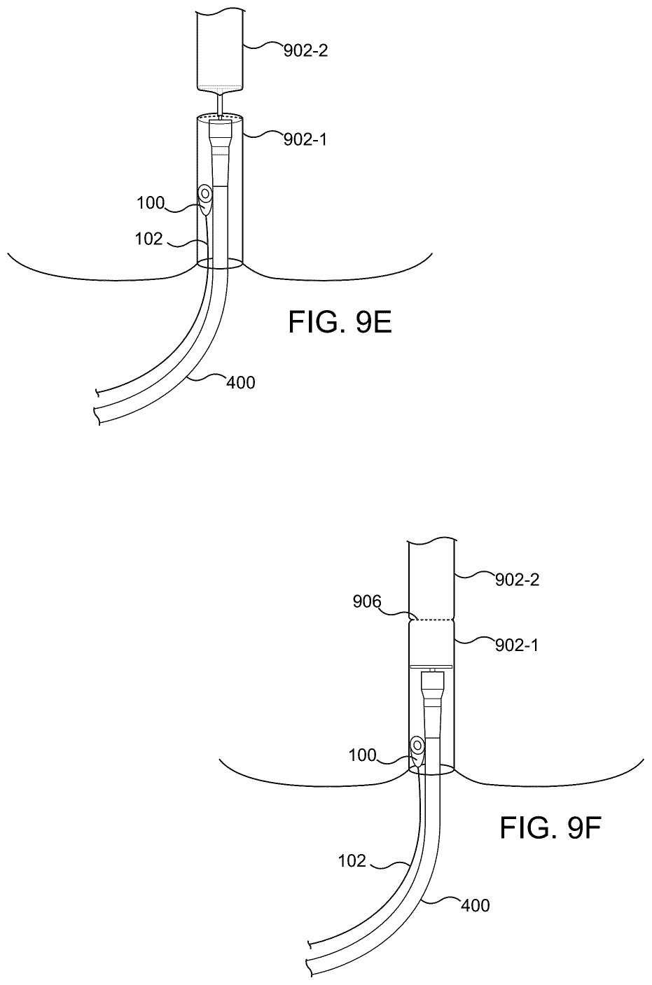

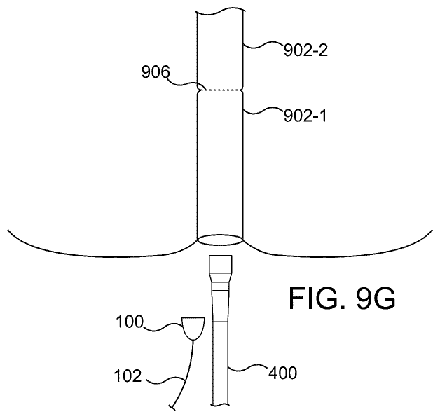

FIG. 9A to 9G depict an example of using the stapler 500 and device 100 in a procedure according to some embodiments. In FIG. 9A, stapler 500 and device 100 have been attached and can be introduced into a structure 902-1. Structure 902-1 may be a tubular structure with an empty cavity. In some embodiments, structure 902-1 may be the intestine and device 100 is introduced through the rectum into the intestine. However, as discussed above, stapler 500 and device 100 may be used in other procedures.

FIG. 9B shows the insertion of device 100 and stapler 500 into structure 902-1 according to some embodiments. Another structure 902-2 is shown. For example, part of an intestine may be removed creating structure 902-2 as one side of the intestine and structure 902-1 as another side of the intestine. An anvil 904 has been inserted into structure 902-2 and secured to structure 902-2, such as via a pursestrings suture. Anvil 904 may be a plate and stem that are used in the stapling process. A staple line is included in structure 902-1 to close structure 902-1 before stapling to structure 902-2.

FIG. 9C depicts example of device 100 being separated from stapler 500 according to some embodiments. As shown, stapler 500 has its blunt edge exposed when device 100 has been removed from the distal end of stapler 500. The removal may be performed by further retracting needle 506 into stapler 500. For example, needle 506 may be fully retracted into stapler 500 thereby releasing needle 506 from recess 104. Other methods of removing device 100 may also be used. However, retracting needle 506 provides a fast and convenient way of removing device 100.

String 102 may be used to pull and retrieve device 100 from the proximal end of device 100. FIG. 9D shows the retrieval of device 100 according to some embodiments. As shown, device 100 has been retrieved to the side of stapler 500 exposing the blunt end of stapler 500, which prepares stapler 500 to perform the stapling process. In some embodiments, string 102 may be used to pull device 100 towards the opening of structure 902-1 past the one end of stapler 500. Device 100 may collapse as it is moved to the side of stapler 500 in a wall of structure 902-1 based on the force applied to device 100 by the wall of structure 902-1 and stapler 500.

Also, as shown, needle 506 may be extended after the removal of device 100. Needle 506 may be guided through the staple line. Then, needle 506 may be inserted into a shaft of anvil 904. The shaft may be configured with an opening and cavity to receive needle 506. Needle 506 may be retracted to abut structure 902-2 and structure 902-1 together.

FIG. 9E depicts the stapling of structure 902-1 and structure 902-2 according to some embodiments. Stapler 500 may use its stapling device to staple structure 902-1 and structure 902-2 together using anvil 904. The edges of structure 902-1 and structure 902-2 may be stapled together. To join the tubular structures 902-1 and 902-2, one end of structure 902-2 is secured about anvil 904 and the other end of structure 902-1 is held in place adjacent to a stapling device (e.g., a staple cartridge) of stapler 500. Once the anvil shaft is secured to stapler 500, the anvil is drawn into close approximation to the stapling cartridge. Stapler 500 is then fired to cause the staples to pass through tissue of both structure 902-1 and structure 902-2, which makes them become formed against the anvil 904. During the firing step, a circular knife may be advanced to cut tissue inside the staple line, thereby establishing a passage between structure 902-1 and structure 902-2. After firing, stapler 500 may be removed by withdrawing the anvil through the staple line.

FIG. 9F shows an example of the stapled structure at 906 according to some embodiments. Structure 902-1 and structure 902-2 are stapled together. Anvil 904 may still be attached to stapler 500, and stapler 500 with anvil 904 can be removed from structure 902-1. Additionally, device 100 may also be removed. For example, stapler 500 may be removed first, which leaves device 100 in structure 902-1 and then device 100 may be removed. Also, device 100 may be removed first as the collapsible feature of device 100 allows it to be retrieved. Then, stapler 500 may be removed. FIG. 9G depicts an example of stapler 500 and device 100 after removal according to some embodiments. Stapler 500 and anvil 904 are removed. Also, device 100 has been removed from structure 902-1. Device 100 may not be collapsed after removal.

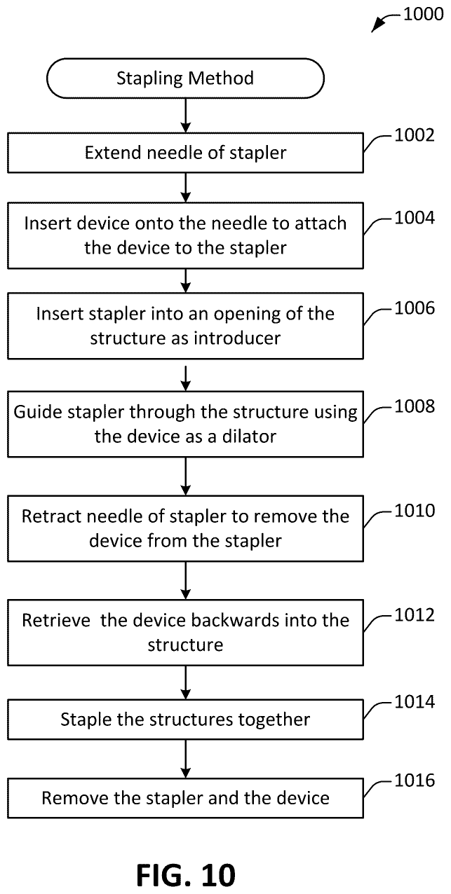

FIG. 10 depicts a simplified flow chart 1000 of a method for performing the stapling of structures according to some embodiments. At 1002, needle 506 of stapler 500 is extended. Then, at 1004, device 100 is inserted onto needle 506 to attach device 100 to stapler 500. As described above, recess 104 may receive and attach to needle 506.

At 1006, stapler 500 is inserted into an opening of structure 902-1 using device 100 as an introducer. At 1008, stapler 500 is guided through a cavity of structure 902-1 using device 100 as a dilator. Device 100 may increase the size of structure 902-1 as it moves through the cavity of structure 902-1. The material of device 100 may not get caught on folds of the cavity. Also, the material of device 100 may be soft, which does not cause trauma to the side of structure 902-1. Device 100 may have a harder core with a softer shell that allows stapler 500 to not get stuck in any creases in the valves of structure 902-1. Device 100 allows the walls of structure 902-1 to expand and also allows the valves to not interfere with the introduction of stapler 500.

At 1010, needle 506 of stapler 500 is retracted to remove device 100 from the stapler. Then, at 1012, device 100 is retrieved backwards into structure 902-1. As discussed above, device 100 may be retrieved to the side of stapler 500 to allow the blunt edge of stapler 500 to be exposed. At 1014, stapler 500 is used to staple the structures 902-1 and 902-2 together. Then, at 1016, stapler 500 and device 100 are removed from structure 902-1.

### CONCLUSION

Device 100 acts as an introducer to introduce stapler 500 and also a dilator to dilate the structure. The material of device 100 allows for the introduction of stapler 500 without damaging structure 902-1. Further, the material of device 100 allows it to collapse to move device 100 to the side of stapler 500 exposing the blunt edge of the stapling device of the stapler 500. Accordingly, many advantages are provided using device 100.

As used in the description herein and throughout the claims that follow, “a,” “an”, and “the” includes plural references unless the context clearly dictates otherwise. Also, as used in the description herein and throughout the claims that follow, the meaning of “in” includes “in” and “on” unless the context clearly dictates otherwise.

The above description illustrates various embodiments along with examples of how aspects of some embodiments may be implemented. The above examples and embodiments should not be deemed to be the only embodiments and are presented to illustrate the flexibility and advantages of some embodiments as defined by the following claims. Based on the above disclosure and the following claims, other arrangements, embodiments, implementations, and equivalents may be employed without departing from the scope hereof as defined by the claims.

What is claimed is:

1. A device comprising:
a first portion with a rounded shape for introduction into an opening of a structure;
a second portion with a recess to receive a needle from an instrument; and
a retrieval mechanism to retrieve the device after removal of the device from the instrument, wherein the device is collapsible in the structure when removed from the instrument after insertion of the instrument and the device into the structure.

2. The device of claim 1, wherein:
the device introduces the instrument into the structure, and
dilates the structure as the instrument is guided through the structure by the device.

3. The device of claim 1, wherein the recess attaches to the needle when the needle is inserted into the recess.

4. The device of claim 1, wherein a cavity inside of the device receives the needle when the needle is inserted into the recess.

5. The device of claim 1, wherein the second portion of the device is coupled to the instrument upon receiving the needle.

6. The device of claim 1, wherein the device is coupled to the instrument by retracting the needle into the instrument.

7. The device of claim 1, wherein the retrieval mechanism is placed through the recess to the first portion.

8. The device of claim 1, wherein:
the device includes a channel from the recess to the first portion, and
the retrieval mechanism is placed through the channel.

9. The device of claim 1, wherein:
the first portion comprises a first material at a tip of the device, and
the first portion comprises a second material on a side of the device.

10. The device of claim 9, wherein the first material is harder than the second material.

11. The device of claim 1, further comprising:
a set of leaflets, wherein the leaflets are collapsible.

12. The device of claim 11, wherein:
a leaflet in the set of leaflets is coupled from a center stud to a side of the device, or
a leaflet in the set of leaflets is coupled from the first portion to the second portion.

13. The device of claim 1, wherein the device is removable from the instrument by retracting the needle into the instrument.

14. The device of claim 1, wherein the instrument performs a stapling procedure while the device is collapsed on a side of the instrument.

15. The device of claim 1, wherein the retrieval mechanism is configured to retrieve the device from the structure.

16. A system comprises:
an instrument comprising:
a needle; and
a device comprising:
a first portion with a rounded shape for introduction into an recess of a structure; and
a second portion with a recess to receive the needle from the instrument, wherein the device is collapsible in the structure when removed from the instrument after insertion of the instrument and the device into the structure.

17. The system of claim 16, wherein:
the instrument comprises a stapling device, and
the stapling device is exposed upon removal of the device from the needle.

18. The system of claim 16, wherein:
the device comprises a retrieval mechanism to retrieve the device after removal of the device from the instrument.

19. The system of claim 16, wherein:
the device introduces the instrument into the structure, and
dilates the structure as the instrument is guided through the structure by the device.

20. A method comprising:
attaching a device to an instrument via a needle of the instrument;
inserting the instrument into a first structure using the device as an introducer;
guiding the instrument through the first structure using the device as a dilator;
removing the device from the needle;
retrieving the device, the device being collapsible when retrieved to a side of the instrument;
stapling the first structure with a second structure; and
removing the device and the instrument.
